(19) United States
(12) Patent Application Publication
(10) Pub. No.: US 2020/0324104 A1
(43) Pub. Date: Oct. 15, 2020

(54) NERVE STIMULATION APPARATUS AND METHOD

(71) Applicant: Lab Schopfergeist AG, Balzers (LI)

(72) Inventors: Chene Labuschagne, Western Cape (ZA); Kevin Duffy, Hampshire (GB); Terrence Howard Henry, Cape Town (ZA)

(21) Appl. No.: 16/305,613

(22) PCT Filed: May 31, 2017
(86) PCT No.: PCT/IB2017/053198

§ 371 Date: Nov. 29, 2018

Related U.S. Application Data
(60) Provisional application No. 62/343,405, filed on May 31, 2016.

(51) Int. Cl. A61N 1/04; A61N 1/32; A61N 1/36; A61N 2/00; A61N 5/06

(52) U.S. Cl. CPC A61N 1/0456; A61N 1/0476; A61N 1/0484; A61N 1/321; A61N 1/36031; A61N 2005/067; A61N 1/36021; A61N 1/36025; A61N 2/006; A61N 5/0622; A61N 2005/0651; A61N 1/36034

(57) ABSTRACT

A nerve stimulator is provided, and in particular a non-invasive nerve stimulator for peripheral nerves, such as the ulnar and/or median nerves. The stimulator may be fitted proximate the left and/or right arm, wrist or hand of a user, to stimulate the median and/or ulnar nerves using electricity, light, sound, magnetic field, vibration or pressure, or any combination of these stimuli. The apparatus takes the form of a standalone band or clasp, or forms part of a fitness tracker band, watch or smartwatch. The nerve stimulator comprises a stimulus generator, to generate any or a combination of the stimuli mentioned above, an applicator to apply the generated stimulus to the arm, wrist or hand of the user, and a controller to control the operation of the stimulus generator and the applicator. In some cases, for stimuli such as light, sound, magnetic field, vibration or pressure, the generator and applicator may be combined into a single component.

### FIELD OF THE INVENTION

THIS INVENTION relates to an apparatus for and method of stimulating nerves in the arm, wrist or hand, and in particular to an apparatus for the neurostimulation of the peripheral nerves of a user, including, but not limited to, the ulnar and/or median nerves.

In an embodiment, the apparatus may take the form of a portable, non-invasive module arranged to stimulate the median and/or ulnar nerves using electricity, light, sound, magnetic field, vibration or pressure, proximate the arm, wrist or hand of a user, which may either take the form of a standalone band or clasp, or which may be integrated into a fitness tracker band, watch or smartwatch.

The potential users of the apparatus (and related methodologies) of the present invention include individual consumers and/or patients, with a view to improving non-medical indications and medical indications.

The non-medical indications include, but are not limited to, sports performance and endurance, including addressing fatigue, alertness and motorskill development. The non-medical indications may further include, but again are not limited to, improving cognitive performance, such as learning, reading, attention or multitasking, weight loss and jet lag, as well as mindfulness.

The medical indications, include, but are not limited to, improving ADHD, depression, epilepsy, insomnia, migraine, anxiety, acute and chronic pain, cardiovascular disorders, movement disorders and functional restoration.

### BACKGROUND OF THE INVENTION

The nervous system consists of the central nervous system and peripheral nervous system. The function of the peripheral nervous system (PNS) is to connect the central nervous system to the limbs and organs.

Various spinal nerves contribute to form nerve plexi-networks of interconnecting nerves. The brachial plexus is one of a few major plexi, and is fed into from five of the 31 spinal nerves. Extending from the brachial plexus into the human forearm are the median and ulnar nerves, as shown in FIG. 1. The median and ulnar nerves form conduits to the brain and autonomic nervous system via the brachial plexus and spinal nerves. Stimulation of these nerves can influence motor/sensory brain functions, as well as the autonomic nervous system.

### SUMMARY OF THE INVENTION

In broad terms, the present invention provides a nerve stimulator, and in particular a non-invasive nerve stimulator for peripheral nerves, such as the ulnar and/or median nerves, which may be fitted proximate the left and/or right arm, wrist or hand of a user, to stimulate the median and/or ulnar nerves using electricity, light, sound, magnetic field, vibration or pressure, or any combination of these stimuli, the apparatus taking the form of a standalone band or clasp, or forming part of a fitness tracker band, watch or smartwatch. The nerve stimulator, or a plurality of stimulators, may be fitted so as to stimulate the user's left arm and/or wrist and/or hand, the user's right arm and/or wrist and/or hand, or the user's left and right arm and/or wrist and/or hand.

In slightly more detail, the nerve stimulator comprises a stimulus generator, to generate any or a combination of the stimuli mentioned above, an applicator to apply the generated stimulus to the arm, wrist or hand of the user, and a controller to control the operation of the stimulus generator and the applicator. In some cases, for stimuli such as light, sound, magnetic field, vibration or pressure, the generator and applicator may be combined into a single component.

In an embodiment, the applicator comprises at least one stimulus interface, which would take various forms, depending on the nature of the stimulus, to apply the stimulus to the user's skin, typically proximate to the anterior or ventral side of the user's arm, wrist or hand.

In an embodiment, the stimulus may be applied either as a continuous waveform, including square, rectangular, sinusoidal or triangular waveforms, or as a series of pulses.

In an embodiment, and depending on the application, the stimulator may include user biometric measuring sensors, which may be embodied in or proximate the applicator (but need not be necessarily), to monitor and determine physiological parameters associated with the user, including any one of, but not limited to, heart rate (HR), heart rate variability (HRV), temperature, SPO2, GSR (galvanic skin response) and inertial measurement.

In an embodiment, the controller may include a user biometric measuring module in communication with the user biometric measuring sensors.

In an embodiment, the controller may include a GSR (galvanic skin response) detection module in communication with a GSR sensor, to measure the electrical conductance of the user's skin and/or a user biometric measuring module.

In an embodiment, controller may include an inertial measurement (IMU) module in communication with a IMU sensor to determine whether the user is engaged in athletic activity.

In some embodiments, the user biometric measuring sensors, for monitoring and determining the physiological parameters associated with the user, and the stimulus interface are spaced apart or segmented by at least 30 degrees around the circumference of the user's arm, wrist or hand.

In an embodiment, the controller includes a stimulus controller module to control the applied stimulation in accordance with a stimulation profile, the stimulation profile defining the applied stimulus in terms of duration and/or frequency and/or intensity/amplitude and/or width.

In an embodiment, and again depending on the application, the stimulator may include a communication module to facilitate communication and data transfer between the nerve stimulator and an external/remote device, including the user's mobile device (i.e. smartphone, tablet or smartwatch) and/or a remote server on the cloud. It is envisaged that the data being transferred from the stimulator may include information regarding the stimulus being applied, including duration, frequency, strength etc. and/or the physiological parameters determined by the biometric measuring sensors. Conversely, it is envisaged that the data being transferred to the stimulator from the external/remote device may include information regarding a variation to the stimulus to be applied (i.e. to implement an amended or adjusted stimulation profile), which the controller may then implement, via the stimulus controller module, in conjunction with the generator and applicator.

In an embodiment, a battery and related battery charger may be connected to the applicator.

### Electrical Stimulus

In the case in which the stimulus is an electrical stimulus, the stimulus generator includes a high voltage generator and a current limiter to generate the electrical stimulus for application via a switching matrix.

In an embodiment, the electrical stimulus comprises a series of electrical pulses, with the stimulus controller module including a switch control module and a pulse control module to control switching of the electrical stimulus, via the switching matrix, in accordance with the stimulation profile.

In an embodiment, the stimulus interface includes at least one switching arrangement connected to the switching matrix to control the operation of the stimulus interface.

In an embodiment, the battery and related battery charger are connected to the applicator, and in particular to each switching arrangement associated with each stimulus interface.

In a first embodiment, the stimulus interface includes at least one electrode pair to send current through the user's tissue in order to stimulate the relevant nerve/s.

In an embodiment, at least one electrode pair has a dual function to enable the electrode pair to provide and receive electrical current.

In an embodiment, the electrode pairs may be arranged into a predetermined configuration to allow targeted stimulation of specific areas of the nervous system. More specifically, a configuration of electrode pairs running parallel may be applied to establish proximity for targeting the ulnar and/or the median nerves, which run alongside the length of the forearm. In one embodiment, two electrode pairs may be provided in a criss-cross pattern to target the ulnar and/or median nerves.

In the case of a series of electrical pulses, each stimulation pulse may comprise a pulse of a single frequency, or may comprise a pulse comprising a combination of two or more pulses of the same or different frequencies.

In an envisaged application, the pulse stimulation profile may have the following parameters:
Frequency range: 0.1 Hz to 1000 Hz
Intensity: 100 uA to 10 mA
Pulsewidth: 1 uS to 100 mS

In a particular embodiment, the stimulation may comprise the sum of two or more frequencies, for example a slow oscillating waveform, having a frequency of around 1 Hz, a medium oscillating waveform superimposed on top of the slow oscillating waveform having a frequency of around 15 Hz, and a high oscillating waveform superimposed on top of the slow waveform pulse and the medium oscillating waveform, having a frequency of between 30 Hz and 50 Hz.

### Magnetic Stimulus

In the case in which the stimulus is a magnetic stimulus, the stimulus generator includes a time varying magnetic field generator and related stimulus interface to induce an electrical current in the user's tissue, in order to stimulate the nerve/s.

### Light Stimulus

In the case in which the stimulus is a light stimulus, the stimulus generator includes a light source, such as a light-emitting diode (LED), or a laser, in order to stimulate the nerve/s. Unlike electrical stimulation, which spreads through tissue and cannot readily be focused, light stimulation offers the advantage of being able to be pinpointed.

### Sound Stimulus

In the case in which the stimulus is sound, the stimulus generator includes a sound generator in order to stimulate the nerve/s.

### Vibration Stimulus

In the case in which the stimulus is a vibration stimulus, the stimulus generator includes a vibration generator, in order to stimulate the nerve/s.

### Pressure Stimulus

In the case in which the stimulus is a pressure stimulus, the stimulus generator includes a vibration generator, in order to stimulate the nerve/s.

In an embodiment, the nerve stimulator may take the form of a portable, wearable stimulator, which may be embodied and/or housed and/or accommodated and/or integrated within and/or secured to (with a clip, for example) a fitness tracker band, clasp, patch, strap, or sleeve or conventional watch. In one version, and with particular reference to watches (typically, upper end, luxury watches) that make use of so-called butterfly clasps comprising a central, curved clipping body, to which adjacent flexible strap portions are hingedly fitted, the nerve stimulator may be integrally fitted, or removably fittable, to the clipping body.

In a first application, the nerve stimulator may take the form of a portable, wearable stimulator, which may be coupled to a smartwatch to form an integral device.

In one particular embodiment, a smartwatch comprises a watch computing device fitted to a strap, typically positioned on the dorsal side of the user's wrist, with the ventral side of the strap comprising the nerve stimulator of the present invention. Although they are separate and distinct components, the communication module of the nerve stimulator enables communications and/or the transfer of data and information between the watch computing device and the nerve stimulator, typically wirelessly. In this application, the watch computing device and the nerve stimulator may be arranged diametrically opposite each other on the strap.

The communication module of the nerve stimulator may be any suitable communication component for both transmitting stimulation data to and receiving biometric data from the processor located on the dorsal side of the wrist. In addition, the pulse control module may also include controller logic to trigger electrical stimulation pulse profiles based on received biometric data from the smart watch.

In a second application, the stimulus controller module applies a stimulation profile based on the physiological parameters received by the user biometric measuring module from the user biometric measuring sensors and/or an indication from the GSR detection module and/or IMU module, so as to define a closed loop bio-feedback arrangement.

In this embodiment, the nerve stimulator may take the form of a wearable band, patch, or strap, of the type described above, which may be worn on the user's arm, wrist or hand.

In one version, the stimulus controller module may compare the received physiological parameters to a predetermined level or profile, and select an alternative stimulation profile (or adjust the existing stimulation profile being used).

The stimulus controller module continues to monitor the physiological parameters, and once the predetermined level or profile of the physiological parameters is achieved, yet another stimulation profile may be selected and applied to maintain the predetermined level or profile of the physiological parameters (or the stimulation may be stopped).

Thus, in a related method, the invention extends to a method of operating a nerve stimulator of the type defined above, the method comprising:
receiving physiological parameters associated with a user;
comparing the received physiological parameters to a predetermined level or profile; and
selecting an alternative stimulation profile, or adjust the existing stimulation profile being used, accordingly.

The method includes continuing to monitor the physiological parameters, and once the predetermined level or profile of the physiological parameters is achieved, the method includes selecting another stimulation profile to maintain the predetermined level or profile of the physiological parameters (or stopping the stimulation).

In a third application, the nerve stimulator may be used in conjunction with an interactive computer cognitive training application, such as a language or education software running on a training computer, which the user may interact with. In this application, a training processing module is provided to co ordinate the application of the stimulus with particular activities or learning elements that the user must engage with, and/or at predetermined times during the training.

In one version, the training processing module may monitor the user's performance during the training, and instruct the stimulus controller module to select a stimulation profile (or select another stimulation profile, if one is already being used.) The training processing module continues to monitor the user's performance, and once a satisfactory performance level has been achieved, yet another stimulation profile may be selected and applied to maintain the user's performance (or the stimulation may be stopped).

In this embodiment, the nerve stimulator may take the form of a wearable band, patch, or strap, of the type described above, which may be worn on the user's arm, wrist or hand, the nerve stimulator being connected to (or at least in communication with) the training processing module (via the communication module and related communication component), which in turn is connected to (or at least in communication with) the training computer.

In a fourth application, the nerve stimulator may be used in conjunction with a visualizing device running on a visualization computer, which the user may interact with to view corresponding anatomical movements during stimulation of the user's median and/or ulnar nerves.

In this application, a visualization module is provided to co ordinate, typically in tandem, the application of the stimulation to the median and/or ulnar nerves with the display on the visualizing device of the movement of a muscle or muscle group associated with the median and/ulnar nerve. The visualizing device may take the form of a virtual reality or ocular feedback device. In an embodiment, the extent and nature of the virtually depicted activity or movement will coincide with the amount of stimulation provided.

In this embodiment, the nerve stimulator may take the form of a wearable band, patch, or strap, of the type described above, which may be worn on the user's arm, wrist or hand, the nerve stimulator being connected to (or at least in communication with) the visualization module (via the communication module and related communication component), which in turn is connected to (or at least in communication with) the visualization computer.

### BRIEF DESCRIPTION OF THE DRAWINGS

FIG. 1 shows an anatomical illustration of the median and ulnar nerves;

FIG. 2 shows a schematic view of a nerve stimulator of the invention, according to an embodiment in which the stimulator takes the form of a wrist band;

FIG. 3 shows a schematic view of a nerve stimulator of the invention, according to an embodiment in which the stimulator is coupled to a smartwatch;

FIG. 4 shows a high level schematic block diagram of some of the possible components of the nerve stimulator of the invention;

FIG. 5 shows a schematic block diagram of one possible version of the nerve stimulator, in which the nerve stimulator makes use of electrical stimulation;

FIG. 6 shows an example pulse stimulation waveform of the type that may be generated by the nerve stimulator of the invention;

FIG. 7 shows an alternative pulse stimulation waveform of the type that may be generated by the nerve stimulator of the invention;

FIG. 8 shows a schematic diagram of the nerve stimulator used in conjunction with an interactive computer cognitive training application;

FIG. 9 shows a schematic diagram of the nerve stimulator used in conjunction with a visualization application; and

FIG. 10 shows a schematic flow chart representing a method of operating a non-invasive nerve stimulator for peripheral nerves, according to a further embodiment of the invention.

### DETAILED DESCRIPTION OF THE INVENTION

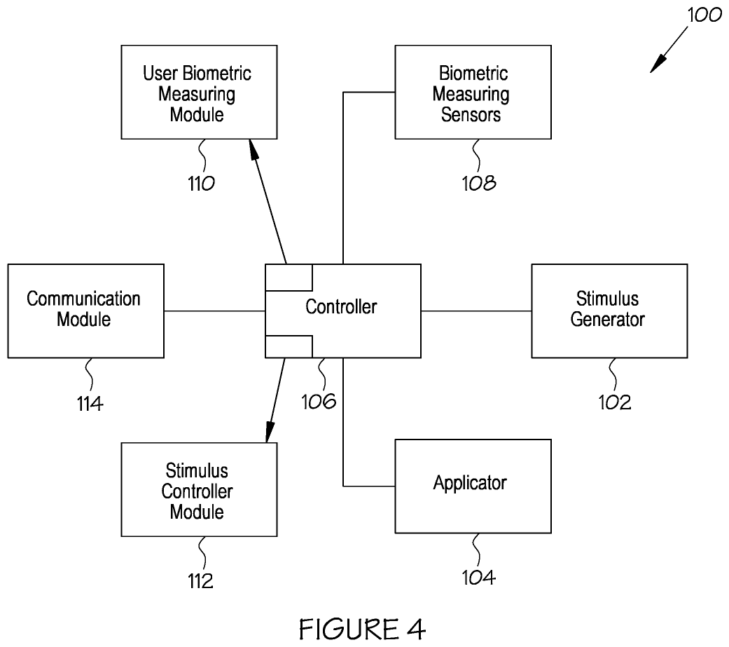

Referring first to FIG. 4, in broad terms, a nerve stimulator 100 is provided, and in particular a non-invasive nerve stimulator for peripheral nerves, such as the ulnar and/or median nerves, which may be fitted proximate the arm, wrist or hand of a user, to stimulate the median and/or ulnar nerves using electricity, light, sound, magnetic field, vibration or pressure, or any combination of these stimuli. The stimulator 100 may take the form of a standalone band or clasp, or forming part of a fitness tracker band, watch or smartwatch.

The nerve stimulator 100 may comprise a stimulus generator 102, to generate any or a combination of the stimuli mentioned above, an applicator 104 to apply the generated stimulus to the arm, wrist or hand of the user, and a controller 106 to control the operation of the stimulus generator 102 and the applicator 104. In some cases, for stimuli such as light, sound, magnetic field, vibration or pressure, the generator 102 and applicator 104 may be combined into a single component.

In an embodiment, the applicator 104 comprises at least one stimulus interface, which could take various forms, depending on the nature of the stimulus, to apply the stimulus to the user's skin, typically proximate to the anterior or ventral side of the user's arm, wrist or hand.

The stimulus may be applied either as a continuous waveform, including square, rectangular, sinusoidal or triangular waveforms, or as a series of pulses.

In an embodiment, and depending on the application, the stimulator 100 may include user biometric measuring sensors 108, which may be embodied in or proximate the applicator 104 (but need not be necessarily), to monitor and determine physiological parameters associated with the user, including any one of, but not limited to, heart rate (HR), heart rate variability (HRV), temperature, SPO2, GSR (galvanic skin response) and inertial measurement.

In an embodiment, the controller 106 may include a user biometric measuring module 110 in communication with the user biometric measuring sensors 108. The controller 106 may include a GSR (galvanic skin response) detection module in communication with a GSR sensor, to measure the electrical conductance of the user's skin and/or a user biometric measuring module. In addition, the controller 106 may include an inertial measurement (IMU) module in communication with an IMU sensor to determine whether the user is engaged in athletic activity.

In some embodiments, the user biometric measuring sensors 108, for monitoring and determining the physiological parameters associated with the user, and the stimulus interface are spaced apart or segmented by at least 30 degrees around the circumference of the user's arm, wrist or hand.

In an embodiment, the controller 106 includes a stimulus controller module 112 to control the applied stimulation in accordance with a stimulation profile, the stimulation profile defining the applied stimulus in terms of duration and/or frequency and/or intensity/amplitude and/or width.

In an embodiment, and again depending on the application, the stimulator 100 may include a communication module 114 to facilitate communication and data transfer between the nerve stimulator 100 and an external/remote device, including the user's mobile device (i.e. smartphone, tablet or smartwatch) and/or a remote server on the cloud.

It is envisaged that the data being transferred from the stimulator 100 may include information regarding the stimulus being applied, including duration, frequency, strength etc. and/or the physiological parameters determined by the biometric measuring sensors 108.

Conversely, it is envisaged that the data being transferred to the stimulator 100 from the external/remote device may include information regarding a variation to the stimulus to be applied (i.e. to implement an amended or adjusted stimulation profile), which the controller 106 may then implement, via the stimulus controller module 112, in conjunction with the generator 102 and applicator 104.

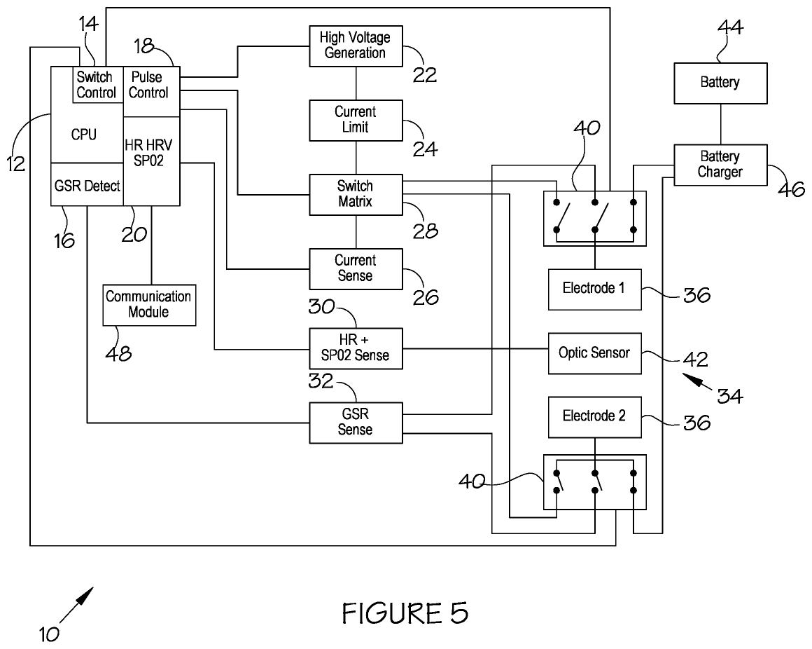

Turning now to FIG. 5, a nerve stimulator 10, and in particular a non-invasive nerve stimulator 10 for electrically stimulating a user's ulnar and/or median nerves, is provided. This may, however, be applied to all peripheral nerves.

The stimulator 10 comprises a controller 12 comprising a switch control module 14, a GSR (galvanic skin response) detection module 16, a pulse control module 18, and a user biometric measuring module 20.

The stimulator 10 further comprises a high voltage generator 22 and current limiter 24 (and related current sensor 26) to generate stimulation pulses for application via a switching matrix 28.

The stimulator 10 further comprises user biometric measuring sensors 30 to determine physiological parameters associated with the user, and which is typically connected to the user biometric measuring module 20.

The stimulator 10 comprises a GSR sensor 32, in communication with the GSR detection module 16, to measure the electrical conductance of the user's skin.

The stimulator 10 comprises an applicator 34, which provides a contact surface for contacting the outer skin surface of the user, comprising a stimulus interface, typically in the form of an electrode pair 36. Clearly, if any of the other stimuli mentioned above, namely light, sound, magnetic field, vibration or pressure, are used, the stimulus interface would differ accordingly.

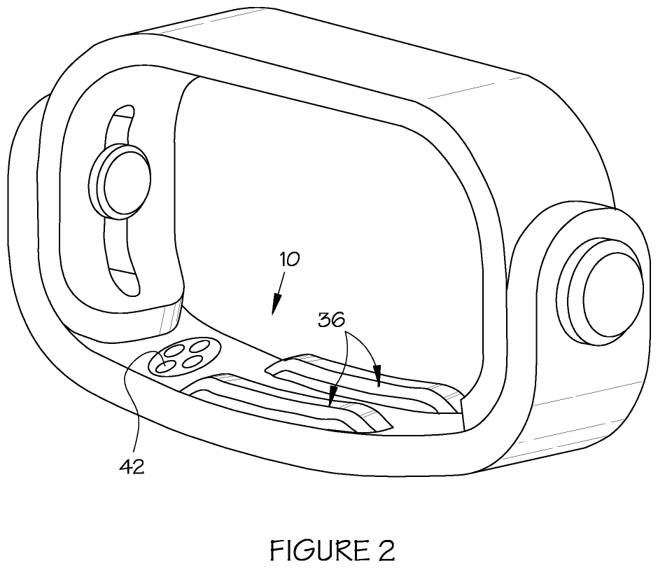

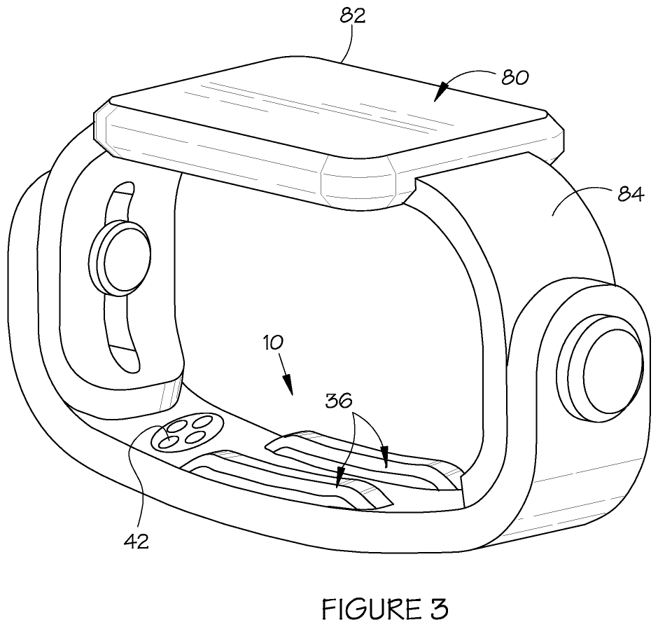

The electrode pair 36, applies the stimulation pulses to the user's skin, typically proximate the ventral side of the user's wrist 38 (as best shown in FIGS. 2 and 3, and which will be described in more detail further below). Although the electrodes of the electrode pair 36 are shown adjacent each other, in one version, only one electrode (e.g. cathode) may be secured to the user's arm, wrist or hand, with the other electrode (e.g. anode) being located on another part of the user's body.

Each electrode 36 has a related switching arrangement 40 connected to the switching matrix 28 to control the operation of the electrode 36.

The applicator 10 further comprises an optic sensor 42 to assist in determining the physiological parameters associated with the user, including heart rate (HR) (using light pulse diodes, for example), heart rate variability (HRV), temperature and SPO2 (to provide an estimate of the user's arterial oxygen saturation).

In an embodiment, a rechargeable battery 44 and related battery charger 46 are connected to the applicator 34, and in particular to each switching arrangement 40 associated with each electrode 36. A design aim of the nerve stimulator 10 of the present invention is to provide a stimulator with sufficiently few components/parameters to enable the stimulator 10 to take the form of a stand-alone device that is powered by the battery 44, which may take the form of a replaceable battery.

In an embodiment, each electrode 36 is a dual function electrode 36 that may provide and receive electrical current. The electrode pair 36, in addition to receiving electrical current to facilitate its operation, as described above, may be arranged to provide electrical charge to charge the rechargeable battery 44.

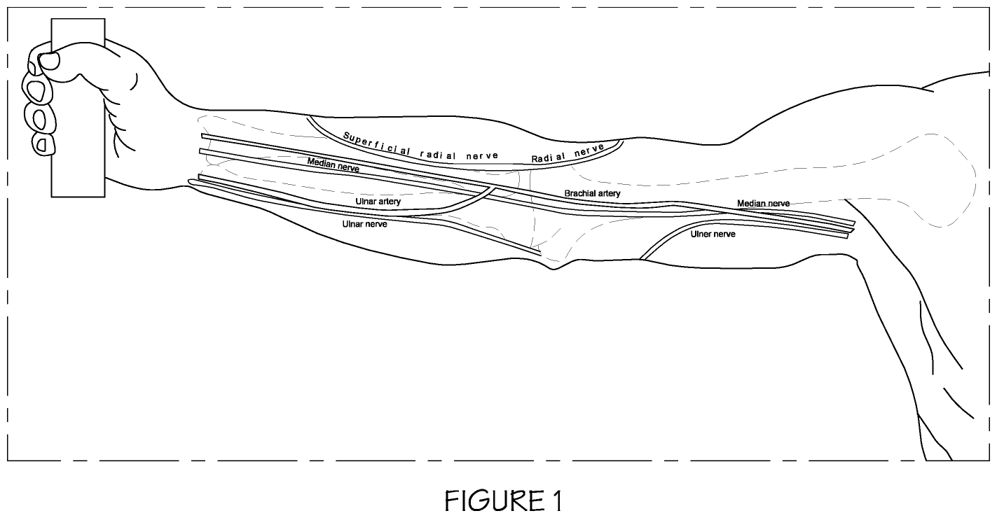

In an embodiment, the applicator 36 comprises a pair of electrodes to apply the stimulation pulses. In an embodiment, the electrodes 36 may be arranged into a predetermined configuration to allow targeted stimulation of specific areas of the nervous system. More specifically, a configuration of parallel electrodes may be applied to establish proximity for targeting the ulnar and the median nerves, which run alongside the length of the forearm (as shown in FIG. 1). In one embodiment, two pairs of electrodes 36 may be provided in a criss-cross pattern to target the ulnar and median nerves.

In some embodiments, the sensor 42, for determining the physiological parameters associated with the user, and the electrodes 36 are spaced apart or segmented by at least 30 degrees around the circumference of the user's wrist 38, as shown in FIGS. 2 and 3.

Although not shown, the nerve stimulator may include an inertial measurement (IMU) module to determine whether the user is engaged in athletic activity.

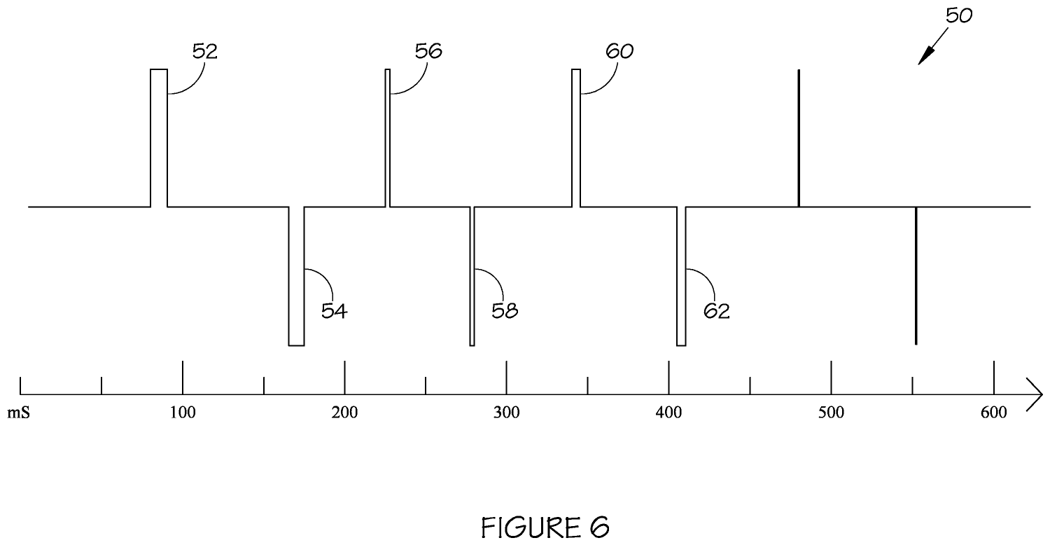

The pulse control module 18 is arranged to stimulate the nerves based on predetermined electrical stimulation patterns, comprising: stimulation session lengths, frequencies, amplitude and pulse width. An example of a stimulation waveform 50 of the type that may be generated by the nerve stimulator 10 of the invention is shown in FIG. 6, comprising:
Positive and negative pulses 52, 54 of a relatively long duration of 15 ms, spaced 90 ms apart;
Positive and negative pulses 56, 58 of a relatively short duration of 5 ms, spaced 60 ms apart;
Positive and negative pulses 60, 62 of a relatively intermediate duration of 10 ms, spaced 70 ms apart; and
Positive and negative pulse bursts 64, 66, spaced 80 ms apart;

Clearly, many other stimulation profiles may be used. In particular, a predetermined profile electrical stimulation may be used to affect certain conditions. In addition, different signal profiles may be useful for affecting different levels of the same condition (mild sleep problems vs. severe insomnia).

It is envisaged that superimposing distinct signals that have uniquely associated parameter profiles may have benefits for affecting a single or variety of conditions. In some embodiments, 3 or more signals may be superimposed upon one another. In yet other embodiments, superimposition may occur between signals of differing stimulation types for example when ultrasound simulation is coupled with non-invasive electrical stimulation. In one version, the pulse stimulation profile may have the following parameters:
Frequency range: 0.1 Hz to 1000 Hz
Intensity: 100 uA to 10 mA
Pulsewidth: 1 uS to 100 mS

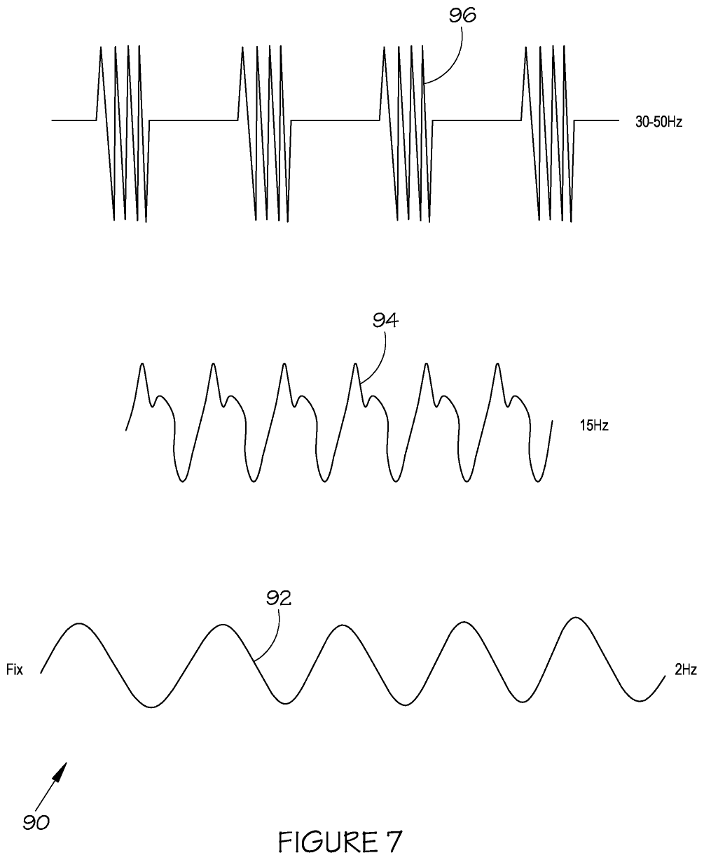

In a particular embodiment, turning now to FIG. 7, the pulse stimulation profile 90 may comprise a slow oscillating pulse 92, having a frequency of around 1 Hz, a medium oscillating pulse 94 superimposed on top of the slow oscillating pulse having a frequency of around 15 Hz, and a high oscillating pulse 96 superimposed on top of the slow oscillating pulse and the medium oscillating pulse, having a frequency of between 30 Hz and 50 Hz.

Turning back to FIG. 5, the nerve stimulator 10 may include a communication module 48 to facilitate communication and data transfer between the nerve stimulator 10 and an external/remote device, including the user's mobile device (i.e. smartphone, tablet or smartwatch) and/or a remote server on the cloud, the communication module 48 including a suitable communication component such as BlueTooth, RFID, NFC, Wi-Fi, ZigBee etc.

Conveniently, the nerve stimulator 10 may take the form of a portable, wearable stimulator, which may be embodied and/or housed and/or accommodated and/or integrated within and/or secured to (with a clip, for example) a band (as shown in FIG. 2), smartwatch (as shown in FIG. 3), patch, strap, or sleeve.

With reference to FIG. 3 in particular, the nerve stimulator 10 may take the form of a portable, wearable stimulator, which may be coupled to a smartwatch 80 to form an integral device. A smartwatch 80 is a computerized wristwatch with extended functionality beyond timekeeping. Modern smartwatches 80 are effectively wearable computers, and as such have significantly expanded functionalities; many run mobile applications using a mobile operating system and in many cases emulate modern mobile telephones/smartphones.

In one particular application, the smartwatch 80 comprises a watch computing device 82 fitted to a contiguous strap 84, typically positioned on the dorsal side of the user's wrist, with the ventral side of the strap 84 comprising the nerve stimulator 10 of the present invention. Although they are separate and distinct components, the communication module 48 of the nerve stimulator 10 enables communications and/or the transfer of data and information between the watch computing device 82 and the nerve stimulator 10, typically wirelessly. In this application, the watch computing device 82 and the nerve stimulator 10 may be arranged diametrically opposite each other on the strap 84. The independent and separate controllers found in the watch computing device 82 and the nerve stimulator 10 allow for efficient coupling between two distinct electronics adorned to a user's wrist.

The communication module 48 of the nerve stimulator may be any suitable communication component (such as for BlueTooth, RFID, NFC, Wi-Fi, ZigBee etc.) for both transmitting stimulation data to and receiving biometric data from the processor located on the dorsal side of the wrist. In addition, the pulse control module 18 may also include controller logic to trigger electrical stimulation pulse profiles based on received biometric data from the smartwatch 80.

In an embodiment, the pulse control module 18 may also include controller logic to trigger electrical stimulation pulse profiles based on the physiological parameters received by the biometric measuring module 20 from the user biometric measuring sensors 30 and/or an indication from the IMU module regarding the user's physical activity, so as to define a closed loop bio-feedback arrangement. This leverages individual responses to stimulation, such as heart rate (HR) or heart rate variability (HRV) responses in the context of a fitness stimulation application, for example. In one particular application, although not limited thereto, this embodiment of the invention relates to the management of the user's heart rate, using stimulation of the median and/or ulnar nerves in a closed loop manner to achieve a desired physiological state.

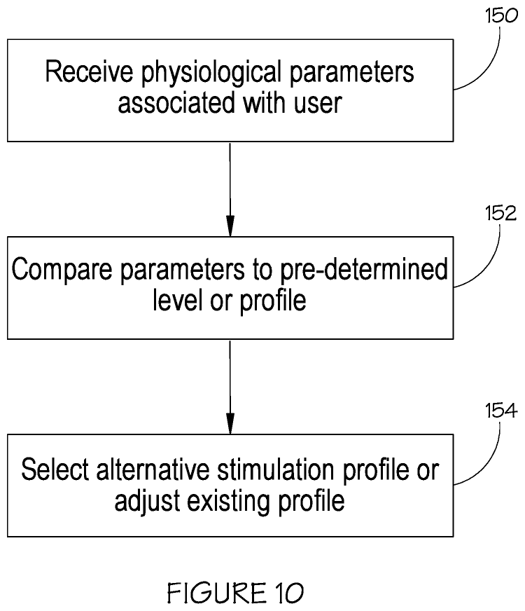

In one version, as shown in FIG. 10, the pulse control module 18 may compare the received physiological parameters to a predetermined level or profile (blocks 150 and 152), and select an alternative electrical stimulation pulse profile (or adjust the existing profile being used), as shown in block 154. The pulse control module 18 continues to monitor the physiological parameters, and once the predetermined level or profile of the physiological parameters is achieved, yet another electrical stimulation pulse profile may be selected and applied to maintain the predetermined level or profile of the physiological parameters (or the stimulation may be stopped).

In this embodiment, the nerve stimulator may take the form of a wearable band, patch, or strap, of the type described above, which may be worn on the user's wrist, as shown in FIG. 2.

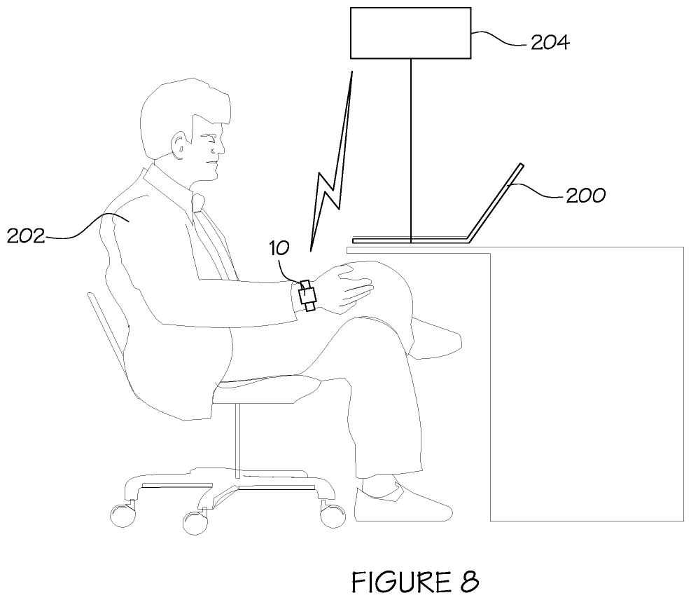

In an embodiment, turning now to FIG. 8, the nerve stimulator 10 may be used in conjunction with an interactive computer cognitive training application, such as a language or education software running on a training computer 200, which the user 202 may interact with. In this application, a training processing module 204 is provided to co ordinate the application of the stimulation pulses with particular activities or learning elements that the user must engage with, and/or at predetermined times during the training. The aim of this particular embodiment is to co ordinate learning, training and educating, either language or cognitive skills, with stimulation of the median and/or ulnar nerves.

In one version, the training processing module 204 may monitor the user's performance during the training, and instruct the pulse control module 18 to select an electrical stimulation pulse profile (or select another electrical stimulation pulse profile, if one is already being used). The training processing module 204 continues to monitor the user's performance, and once a satisfactory performance level has been achieved, yet another electrical stimulation pulse profile may be selected and applied to maintain the user's performance (or the stimulation may be stopped).

Thus, in use, the training processing module 204 may identify previously failed attempts at questions or levels presenting difficulty to the user 202, and apply an appropriate amount of stimulation leading up to or at the time of the user 202 facing the challenging questions or levels.

In this embodiment, the nerve stimulator 10 may take the form of a wearable band, patch, or strap, of the type described above, which may be worn on the user's arm, wrist or hand, the nerve stimulator 10 being connected to (or at least in communication with) the training processing module 204 (via the communication module 48 and related communication component), which in turn is connected to (or at least in communication with) the training computer 200.

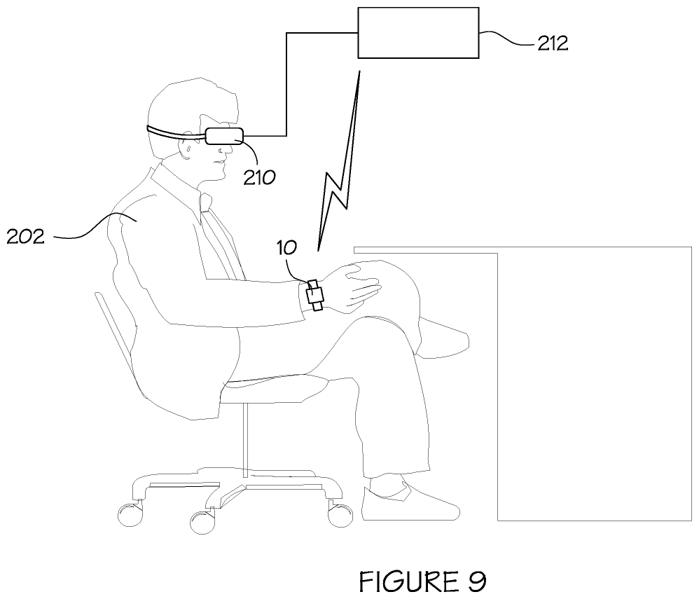

Turning now to FIG. 9, in another embodiment, the nerve stimulator 10 may be used in conjunction with a visualizing device 210 running on a visualization computer, which the user may interact with to view (in the context of a virtual reality experience) corresponding anatomical movements during stimulation of the user's median and/or ulnar nerves. It is believed that stimulating the median nerve in tandem with visual perception of associated muscle movements will trigger pathways in the brain and regenerate lost motor pathways along the nervous system chain that connect the motor cortex through the central nervous system into the median and/or ulnar systems of the peripheral nervous system.

In this application, a visualization module 212 is provided to coordinate, typically in tandem, the application of the stimulation pulses to the median and/or ulnar nerves with the display on the visualizing device 210 of the movement of a muscle or muscle group associated with the median and/ulnar nerve. The visualizing device 210 may take the form of a virtual reality or ocular feedback device.

For example, in one version, the visualization module 212 may coordinate a virtually depicted movement or activity of a user's hand with electrical stimulation of associated anatomical locations. In an embodiment, the extent and nature of the virtually depicted activity or movement will coincide with the amount of stimulation provided.

In this embodiment, the nerve stimulator 10 may take the form of a wearable band, patch, or strap, of the type described above, which may be worn on the user's arm, wrist or hand, the nerve stimulator 10 being connected to (or at least in communication with) the visualization module 210 (via the communication module 48 and related communication component), which may in turn be connected to (or at least in communication with) the visualization computer.

The nerve stimulator 10 of the present invention aims to reduce the number of components/parameters to yield a smaller stimulation module that can be incorporated into a band or wristwatch, preferably to be situated on the dorsal side of the arm, wrist or hand. Such a device, in one embodiment, can send and receive signals with a smartwatch to which it is appended (or another local device such as a tablet or smart phone via Bluetooth) and may be internet connected for direct transceiving of data with a remote server via the cloud. In one embodiment, the stimulation module is embedded within the band clasp that makes contact with the wrist underside.

Over and above the applications described above, the nerve stimulator may be arranged to act as an alarm or notification system. For example, the user may set a wake-up time, typically via a device that is wirelessly connected to the stimulator. At the desired wake-up time, the nerve stimulator may impart a gradual stimulation to activate the user's nervous system and thus awaken the user. The alarm may also provide a notification means, such that the user is alerted when detected biometrics reach certain levels e.g., notify the user has low oxygen (from pulse oximeter), low/high heart rate (from a heart rate monitor), etc. In some embodiments, the alarm system may be tied to GPS/proximity centre such that the user is alerted when he/she has gone off course from their intended destination or location.

Nerve stimulation can facilitate and optimize performance by direct sensory modulation of the sensory motor cortex, indirectly it might have an effect on heart rate variability (HRV) by modulating autonomic functioning and promoting a balance between the sympathetic and parasympathetic systems. By receiving sensory input from the median nerve at the same time a individual is performing a particular form of physical activity, the primary sensory cortex (S1) and the primary motor cortex (M1) will be engaged in the processing of such information through co-activation, this might facilitate plasticity at central level, but it can also improve motor output so the physical activity being performed benefits as the cortex is being primed by the combination of sensory processing. The effects of stimulating the median nerve and/or the ulnar nerve at S1 may also have an effect on pain processing, during exercise and as a result of different factors, pain can be a consequence of prolonged endurance, the modulation from thalamo-cortical circuits due to the stimulation, might increase pain threshold. Depending on the frequencies used, the endogenous opiod system can be stimulated to release enkephalins and endorphins at central and spinal level, thus helping to ease pain.

A specific set of parameters can be used with stochastic properties, where the frequencies generated ranges from 0.5 to 100 Hz and not limited to higher ranges (no more 1000 Hz). The current is delivered in a range of 1 to 6 mA fluctuating over time and in a series of mixed pulses variable characteristics (quadratic, triangular or sinusoidal). Stimulation of the median nerve and/or the ulnar nerve can be coupled with monitoring functions such as heart rate (HR), pulse, and HRV. It can also be used in combination with a system providing biofeedback for heart and breathing capabilities, thus, if integrated stimulation of the median nerve and/or the ulnar nerve can be included in a closed-loop system for stimulation and physiological entrainment. Because the activation of S1 and its close relationship with the fronto-temporal network, attention can be enhanced by stimulation of the median nerve and/or the ulnar nerve, as in the case for HR or HRV, monitoring of cognitive performance through cognitive testing is feasible as well. Sustained attention in specific cognitive tasks will be the fundament for the development of a closed-loop system for cognitive performance. This makes stimulation of the median nerve and/or the ulnar nerve suitable for cognitive and behavioral interventions aimed to optimize learning, reading, attention or multitasking.

In addition, this invention suggests a co-activation process. In particular, the neural level, can be understood as a way to engage a particular neural system that is already being active, in the processing of another type of information coming from another neural unit, these units interact creating thus a network that the two systems form. This shared and simultaneous activation can boost the processing of the entire network. Behaviorally, this can be applied to facilitate the effects of one technique or method by improving the integration and processing of such information.

1. A non-invasive nerve stimulator for peripheral nerves, including the ulnar or median nerves, to be fitted proximate the left or right arm, wrist or hand of a user, to stimulate the median or ulnar nerves using at least one stimulus, the nerve stimulator comprising:
a stimulus generator, to generate at least one stimulus;
an applicator to apply the generated stimulus to the arm, wrist or hand of the user, the applicator comprising at least one stimulus interface to apply the stimulus to the user's skin, proximate to the anterior or ventral side of the user's arm, wrist or hand; and
a plurality of user biometric measuring sensors, embodied in or proximate the applicator, to monitor and determine physiological parameters associated with the user, including heart rate (HR), heart rate variability (HRV), temperature, SPO2, GSR (galvanic skin response) and inertial measurement;
a controller to control the operation of the stimulus generator and the applicator, the controller including a stimulus controller module to control the applied stimulation in accordance with a stimulation profile, the stimulation profile defining the applied stimulus in terms of duration or frequency or intensity or amplitude or width, wherein the stimulus controller module applies a stimulation profile based on the physiological parameters received from the user biometric measuring sensors, so as to define a closed loop feedback arrangement, wherein the stimulus controller module compares the received physiological parameters to a predetermined level or profile, and either selects an alternative stimulation profile or adjusts the existing stimulation profile being used.

2. The nerve stimulator of claim 1, wherein the stimulus controller module continues to monitor the physiological parameters, and once the predetermined level or profile of the physiological parameters is achieved, yet another stimulation profile may be selected and applied to maintain the predetermined level or profile of the physiological parameters or the stimulation is stopped.

3. The nerve stimulator of claim 1, wherein the controller includes:
a GSR (galvanic skin response) detection module in communication with a GSR sensor, to measure the electrical conductance of the user's skin; and
an inertial measurement (IMU) module in communication with a IMU sensor to determine whether the user is engaged in physical activity.

4. The nerve stimulator of claim 1, wherein the user biometric measuring sensors, for monitoring and determining the physiological parameters associated with the user, and the stimulus interface are spaced apart or segmented by at least 30 degrees around the circumference of the user's arm, wrist or hand.

5. The nerve stimulator of claim 1, wherein the stimulator includes a communication module to facilitate communication and data transfer between the nerve stimulator and an external or remote device, including the user's mobile device or a remote server, wherein the data transferred from the stimulator includes information regarding the stimulus being applied, including duration, frequency and strength or the physiological parameters determined by the biometric measuring sensors and wherein the data transferred to the stimulator from the external or remote device includes information regarding a variation to the stimulus to be applied, to implement an amended or adjusted stimulation profile, which the controller can then implement, via the stimulus controller module, in conjunction with the generator and applicator.

6. The nerve stimulator of claim 1, wherein the stimulator or a plurality of stimulators is/are fitted so as to stimulate the user's left arm or wrist or hand, the user's right arm or wrist or hand, or the user's left and right arm or wrist or hand, and
wherein the at least one stimulus includes electricity, light, sound, magnetic field, vibration or pressure, or any combination of these stimuli, with the stimulus being applied either as a continuous waveform, including square, rectangular, sinusoidal or triangular waveforms, or as a series of pulses.

7. (canceled)

8. The nerve stimulator of claim 6, wherein for an electrical stimulus, the stimulus generator includes a high voltage generator and a current limiter to generate the electrical stimulus for application via a switching matrix, wherein the electrical stimulus comprises a series of electrical pulses, with the stimulus controller module including a switch control module and a pulse control module to control switching of the electrical stimulus, via the switching matrix, in accordance with the stimulation profile, and wherein the stimulus interface includes at least one switching arrangement connected to the switching matrix to control the operation of the stimulus interface.

9. The nerve stimulator of claim 8, wherein a battery and related battery charger are connected to each switching arrangement associated with each stimulus interface, wherein the stimulus interface includes at least one electrode pair to send current through the user's tissue in order to stimulate the relevant nerve(s), and wherein at least one electrode pair has a dual function to enable the electrode pair to provide and receive electrical current with the electrode pairs being arranged into a predetermined configuration to allow targeted stimulation of specific areas of the nervous system, with the electrode pair either running parallel to each other for targeting the ulnar or the median nerves, which run alongside the length of the forearm, or being provided in a crisscross pattern to target the ulnar or median nerves.

10. (canceled)

11. The nerve stimulator of claim 8, wherein in the case of a series of electrical pulses, each stimulation pulse may comprise a pulse of a single frequency, or may comprise a pulse comprising a combination of two or more pulses of the same or different frequencies, wherein the pulse stimulation profile is in the frequency range of between 0.1 Hz to 1000 Hz, has an intensity of between 100 uA to 10 mA and a pulse width of between 1 uS to 100 mS, wherein the stimulation comprises the sum of two or more frequencies, including a slow oscillating waveform, having a frequency of around 1 Hz, a medium oscillating waveform superimposed on top of the slow oscillating waveform having a frequency of around 15 Hz, and a high oscillating waveform superimposed on top of the slow waveform pulse and the medium oscillating waveform, having a frequency of between 30 Hz and 50 Hz.

12. (canceled)

13. The nerve stimulator of claim 6, wherein:
for a magnetic stimulus, the stimulus generator includes a time varying magnetic field generator and related stimulus interface to induce an electrical current in the user's tissue, in order to stimulate the nerve(s);
for light stimulus, the stimulus generator includes a light source, selected from a group comprising a light-emitting diode (LED) and a laser, in order to stimulate the nerve(s);
for a sound stimulus, the stimulus generator includes a sound generator in order to stimulate the nerve(s);
for a vibration stimulus, the stimulus generator includes a vibration generator, in order to stimulate the nerve(s); and
for a pressure stimulus, the stimulus generator includes a vibration generator, in order to stimulate the nerve(s).

14. The nerve stimulator of claim 1, wherein the nerve stimulator takes the form of a portable, wearable stimulator, embodied or housed or accommodated or integrated within or secured to a fitness tracker band, clasp, patch, strap, or sleeve or watch or smartwatch, and
wherein the watch makes use of a clasp arrangement comprising a central, curved clipping body, to which adjacent flexible strap portions are hingedly fitted, with the nerve stimulator being integrally fitted, or removably fittable, to the clipping body.

15. (canceled)

16. A smartwatch arrangement comprising:
a watch device fitted to a strap, positioned on the dorsal side of a user's wrist in use, the watch device including a computing device and a plurality of user biometric measuring sensors to monitor and determine physiological parameters associated with the user, including heart rate (HR) or heart rate variability (HRV) or temperature or SPO2 or GSR (galvanic skin response) or inertial measurement; and
a non-invasive nerve stimulator for peripheral nerves including the user's ulnar or median nerves, provided on the ventral side of the strap, in use, to stimulate the median or ulnar nerves using at least one stimulus, the nerve stimulator comprising:
a stimulus generator, to generate at least one stimulus;
an applicator to apply the generated stimulus to the arm, wrist or hand of the user, the applicator comprising at least one stimulus interface to apply the stimulus to the user's skin, proximate to the anterior or ventral side of the user's arm, wrist or hand; and
a controller to control the operation of the stimulus generator and the applicator, the controller including a stimulus controller module to control the applied stimulation in accordance with a stimulation profile, the stimulation profile defining the applied stimulus in terms of duration or frequency or intensity or amplitude or width, wherein the stimulus controller module applies a stimulation profile based on the physiological parameters received from the user biometric measuring sensors, so as to define a closed loop feedback arrangement, wherein the stimulus controller module compares the received physiological parameters to a predetermined level or profile, and either selects an alternative stimulation profile or adjusts the existing stimulation profile being used.

17. The smartwatch arrangement of claim 16, wherein the stimulus controller module continues to monitor the physiological parameters, and once the predetermined level or profile of the physiological parameters is achieved, yet another stimulation profile may be selected and applied to maintain the predetermined level or profile of the physiological parameters or the stimulation is stopped.

18. The smartwatch arrangement of claim 16 wherein the stimulator includes a communication module to facilitate wireless communication and data transfer between the nerve stimulator and the watch computing device, wherein the data transferred from the stimulator includes information regarding the stimulus being applied, including duration, frequency and strength or the physiological parameters determined by the biometric measuring sensors and wherein the data transferred to the stimulator from the watch computing device includes information regarding a variation to the stimulus to be applied, to implement an amended or adjusted stimulation profile, which the controller can then implement, via the stimulus controller module, and
wherein the watch computing device and the nerve stimulator are arranged diametrically opposite each other on the strap, with the communication module of the nerve stimulator comprising a communication component for both transmitting stimulation data to and receiving biometric data from the watch computing device.

19. (canceled)

20. A method of operating a non-invasive nerve stimulator for peripheral nerves, including the ulnar or median nerves, to stimulate the median or ulnar nerves using at least one stimulus, the nerve stimulator comprising a stimulus generator, to generate at least one stimulus according to a stimulation profile, an applicator to apply the generated stimulus to the arm, wrist or hand of the user, and a controller to control the operation of the stimulus generator and the applicator, the method comprising:
receiving physiological parameters associated with a user;
comparing the received physiological parameters to a predetermined level or profile;
selecting an alternative stimulation profile, or adjust the existing stimulation profile being used, accordingly; and
monitoring the user's interaction with an interactive computer training application, and coordinating the application of the stimulus with particular activities or learning elements that the user must engage with or at predetermined times during the training.

21. The method of claim 20, including the step of continuing to monitor the physiological parameters, and once the predetermined level or profile of the physiological parameters is achieved, the method includes selecting another stimulation profile to maintain the predetermined level or profile of the physiological parameters, or stopping the stimulation.

22. The method of claim 21, which includes the steps of:
monitoring the user's performance during the training, and instructing the stimulus controller module to select a stimulation profile or to select another stimulation profile, if one is already being used;
continuing to monitor the user's performance, and once a predetermined performance level has been achieved, selecting another stimulation profile or stopping the stimulation; and
monitoring the user's interaction with a visualizing device running on a visualization computer, which the user may interact with to view corresponding anatomical movements during stimulation of the user's median or ulnar nerves.

23-24. (canceled)

25. A training system comprising:
a non-invasive nerve stimulator for peripheral nerves, including the ulnar or median nerves, to stimulate the median or ulnar nerves using at least one stimulus, the nerve stimulator comprising a stimulus generator, to generate at least one stimulus according to a stimulation profile, an applicator to apply the generated stimulus to the arm, wrist or hand of the user, and a controller to control the operation of the stimulus generator and the applicator; and
a processor arranged:
receive physiological parameters associated with a user;
compare the received physiological parameters to a predetermined level or profile;
select an alternative stimulation profile, or adjust the existing stimulation profile being used, accordingly; and
monitor the user's interaction with an interactive computer training application, and coordinating the application of the stimulus with particular activities or learning elements that the user must engage with or at predetermined times during the training.

26. The system of claim 25, wherein the processor is further arranged to continue monitoring the physiological parameters, and once the predetermined level or profile of the physiological parameters is achieved, select another stimulation profile to maintain the predetermined level or profile of the physiological parameters, or stop the stimulation, and
wherein the processor is further arranged to monitor the user's performance during the training, and instruct the stimulus controller module to select a stimulation profile or to select another stimulation profile, if one is already being used.

27. (canceled)

28. The non-invasive nerve stimulator of claim 1, for use in improving or addressing non-medical indications, including sports performance and endurance, fatigue, alertness, motorskill development, cognitive performance, including learning, reading, attention or multitasking, weight loss and jet lag, and mindfulness, or improving or addressing medical indications, including ADHD, depression, epilepsy, insomnia, migraine, anxiety, acute and chronic pain, cardiovascular disorders, movement disorders and functional restoration.

29. (canceled)
